(19) United States
(12) Patent Application Publication
(10) Pub. No.: US 2025/0134615 A1
(43) Pub. Date: May 1, 2025

(54) SET FOR TREATING THE VASCULATURE TO REDUCE OR BLOCK BLOOD FLOW

(71) Applicant: DePuy Synthes Products, Inc., Raynham, MA (US)

(72) Inventors: Masood SIDDIQUI, Irvine, CA (US); Lalith WIJERATNE, Davie, FL (US); Michael SIMONDS, Plymouth, MA (US); Chelsea VIJIL, Miami, FL (US)

(73) Assignee: DePuy Synthes Products, Inc., Raynham, MA (US)

(21) Appl. No.: 18/384,211

(22) Filed: Oct. 26, 2023

(51) Int. Cl. A61B 50/33; A61B 50/30; A61K 31/275; A61K 33/00

(52) U.S. Cl. CPC A61B 50/33; A61K 31/275; A61K 33/00; A61B 2050/3008

(57) ABSTRACT

A set for treating the vasculature to reduce or block blood flow includes a tray having at least three sections and a center recess configured to receive a mixing container. A sealed vial containing an ethiodized oil is disposed in a first section of the tray. A mixing container is disposed in a first section of the tray. A sealed tube containing a predetermined amount of n-Butyl Cyanoacrylate (“n-BCA”) is disposed in a second section of the tray. The sealed tube has an external thread. A self-piercing luer cap is disposed in the third section of the tray. The cap has an internal thread that mates with the external thread of the sealed tube of n-BCA. A luer lock syringe is disposed in one of the at least three sections of the tray.

### FIELD

The disclosure relates to a set for treating the vasculature to reduce or block blood flow. In particular, this disclosure relates to an endovascular medical procedural set for treating lesions in the peripheral and neuro vasculature.

### BACKGROUND

Trans-arterial embolization is one of the useful methods for the treatment of brain arteriovenous malformations (AVMs) or other locations in the vasculature to reduce or block blood flow. Various kinds of fluid embolic materials have been used for embolization of AVMs. Isobutyl-2-cyanoacrylate (IBCA) was initially used, then it has been replaced by n-Butyl Cyanoacrylate (hereinafter “n-BCA”) in the mid-1980s. Glues such as n-BCA or other cyanoacrylate glues, other agents such as copolymers like Onyx, Squid and PHIL, or other particles to occlude a vessel have also been used. Although it is very difficult to handle a pure form of n-BCA, polymerization time of n-BCA can be prolonged by addition of io-phenydylate or ethiodized oil, which are oil-based radiographic contrast agents and/or a glacial acetic acid. Using these methods, polymerization time can range from between approximately 0.2 and approximately 8.5 seconds.

Because the mixed n-BCA and ethiodized oil or io-phenydylate results in a mixture that polymerizes relatively quickly, there is a need in the art for a set containing all the items needed to mix the n-BCA and ethiodized oil or io-phenydylate with efficiency so that the resulting mixture can be assembled, mixed and then place in a syringe to be injected into a catheter to be delivered to the desired site in the vasculature.

### SUMMARY

A set for treating the vasculature to reduce or block blood flow in accordance with the present invention includes a tray having at least three sections and a center recess configured to receive a mixing container. A sealed vial containing an ethiodized oil is disposed in a first of the three sections of the tray. At least one syringe is disposed in one of the at least three sections the tray. A first needle is disposed in one of the at least three sections the tray. A mixing container is disposed in a first of the at least three sections of the tray. A sealed tube contains a predetermined amount of n-Butyl Cyanoacrylate (“n-BCA”). The sealed tube is disposed in a second of the at least three sections of the tray. The sealed tube has an external thread. A self-piercing luer cap is disposed in one of the at least three sections of the tray. The cap has an internal thread that mates with the external thread of the sealed tube of n-BCA. A luer lock syringe is disposed in one of the at least three sections of the tray.

### BRIEF DESCRIPTION OF THE DRAWINGS

This invention will be more fully understood from the following detailed description taken in conjunction with the accompanying drawings, in which:

FIG. 1 is a perspective view of a box containing the procedural set of the present invention.

FIG. 2 is a perspective view of the box of FIG. 1 shown being opened to permit access to the procedural set of the present invention.

FIG. 3 is a perspective view of the set showing a flexible tray cover that seals the contents of the set and being partially removed thereof to partially expose the retainer cover lid.

FIG. 4 is a top plan view of the retainer cover lid thereof.

FIG. 5 is a cross-sectional view taken from lines 5-5 of FIG. 4.

FIG. 6 is an exploded side view of a flexible tray cover, a retainer cover lid, an inner tray, an outer tray, an instruction card and the box.

FIG. 7A is a top plan view of a first side of the instruction card.

FIG. 7B is a top plan view of a second side of the instruction card.

FIG. 8 is a top plan view of the inner tray.

FIG. 9 is a top plan view of the inner tray with some contents thereof removed from the inner tray.

FIG. 10 is a cross-sectional view of a cavity of the inner tray shown with a solution of 5% Dextrose Solution in the cavity.

FIG. 11 is a top plan view of the inner tray with a beaker in the center of the tray.

FIG. 12 is a cross-sectional view taken along lines 12-12 of FIG. 11.

FIG. 13 is a partial cross-sectional view of the inner tray showing the vial of an ethiodized oil with a syringe and needle being used to remove some of the ethiodized oil from the vial.

FIG. 14 is a cross-sectional view of the inner tray and outer tray showing the syringe of FIG. 13 depositing a pre-determined amount of ethiodized oil into the beaker.

FIG. 15 is a perspective view showing a previously sealed package opened and removing a container of tantalum (“Ta”) powder.

FIG. 16 is a cross-sectional view of the inner tray and outer tray showing the Ta powder being added to the ethiodized oil in the beaker.

FIG. 17 shows the needle of the syringe being used to mix the Ta powder and ethiodized oil in the beaker.

FIG. 18 is an exploded view of a tube containing a predetermined amount of n-Butyl Cyanoacrylate (“n-BCA”), a self-piercing luer cap and a luer lock syringe.

FIG. 19 is side view of a user withdrawing a pre-determined amount of n-BCA from the tube of n-BCA into the luer lock syringe.

FIG. 20 shows a partial cross-sectional view of the inner tray showing the luer lock syringe of n-BCA being added to the mixture of ethiodized oil and Ta powder and being mixed by the needle of the luer lock syringe.

FIG. 21 is a cross-sectional view of the inner tray and outer tray showing a syringe and needle removing a pre-determined amount of mixed n-BCA, ethiodized oil and Ta powder into the syringe.

FIG. 22 is a perspective view of a user injecting the pre-determined amount of mixed n-BCA, ethiodized oil and Ta powder into a catheter to be delivered to site to be treated within the vasculature.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 81% to 99%.

As discussed herein, the terms “distal” or “proximal” are used in the following description with respect to a position or direction relative to the treating physician or medical interventionalist. “Distal” or “distally” are a position distant from or in a direction away from the physician or interventionalist. “Proximal” or “proximally” or “proximate” are a position near or in a direction toward the physician or medical interventionist. The terms “occlusion”, “clot”, “thrombus” or “blockage” are used interchangeably.

As discussed herein, a “subject” or “patient” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

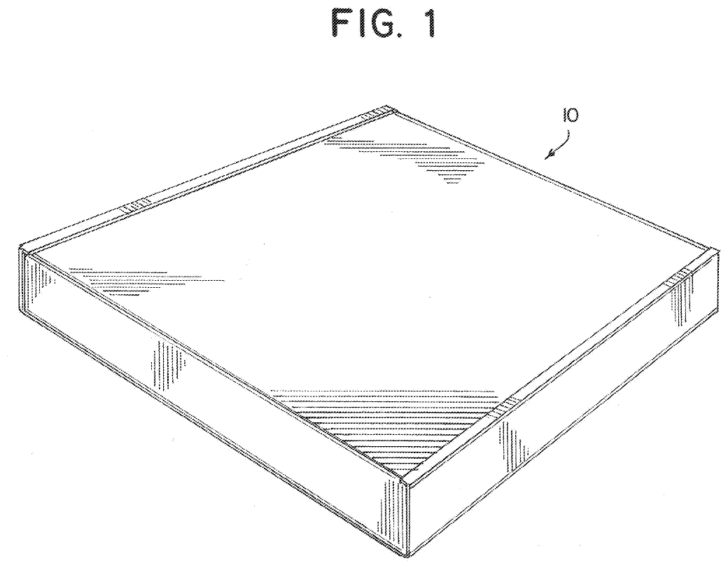

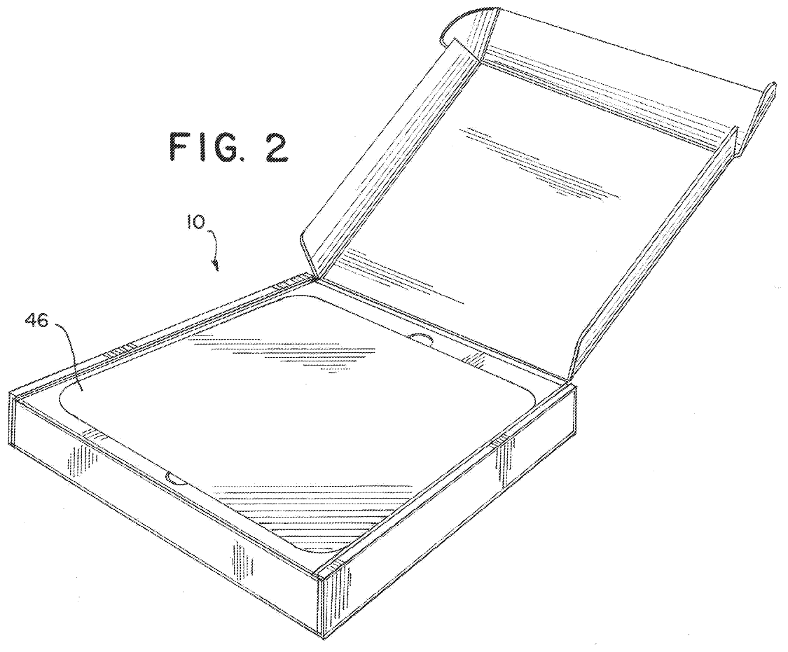

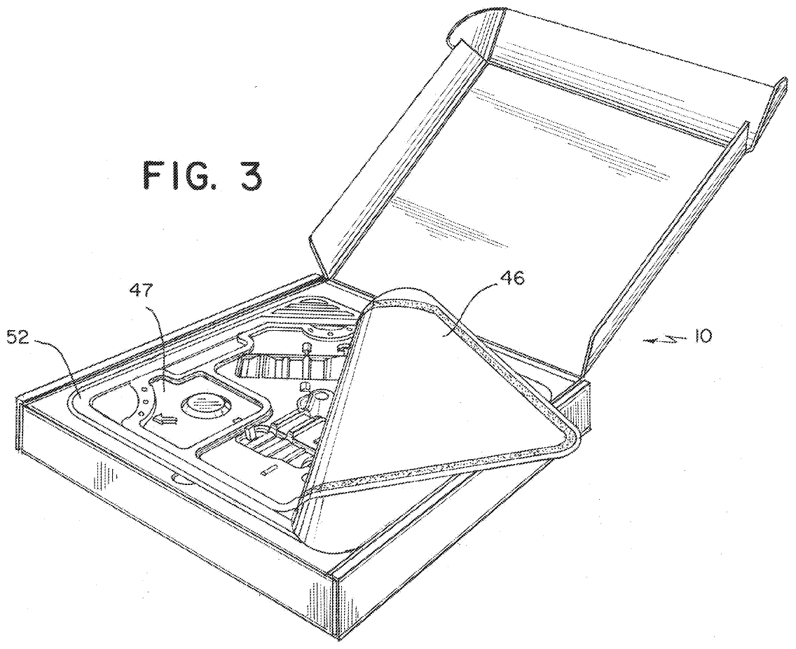

Referring now to FIGS. 1-11, a set 10 for treating the vasculature to reduce or block blood flow is disclosed. Set 10 is illustrated in FIG. 1 being disposed in a shipping box 11. In FIG. 2, box 11 is opened and set 10 is shown with a cover 46 connected to a tray 12 of set 10. With cover 46 connected to tray 12, tray 12 is sealed and the tray contents are configured to be sterilized. Once sterilized, the sealed tray 12 can be shipped in box 11 to the desired site ready to be opened and used at the discretion of the user. As shown in FIG. 3, a user can remove cover 46 from an outer tray 21 to expose the contents of set 10.

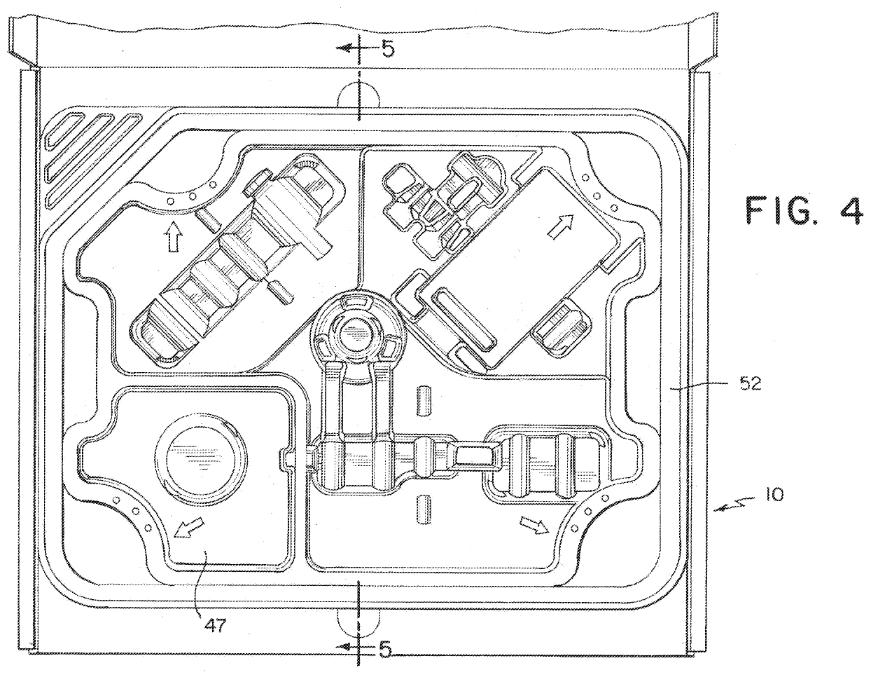

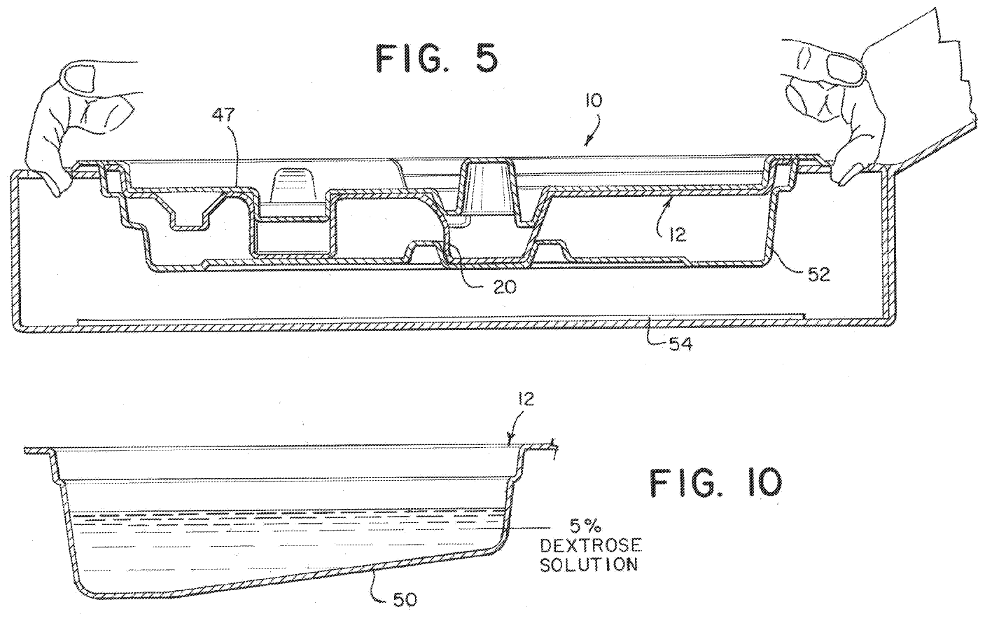

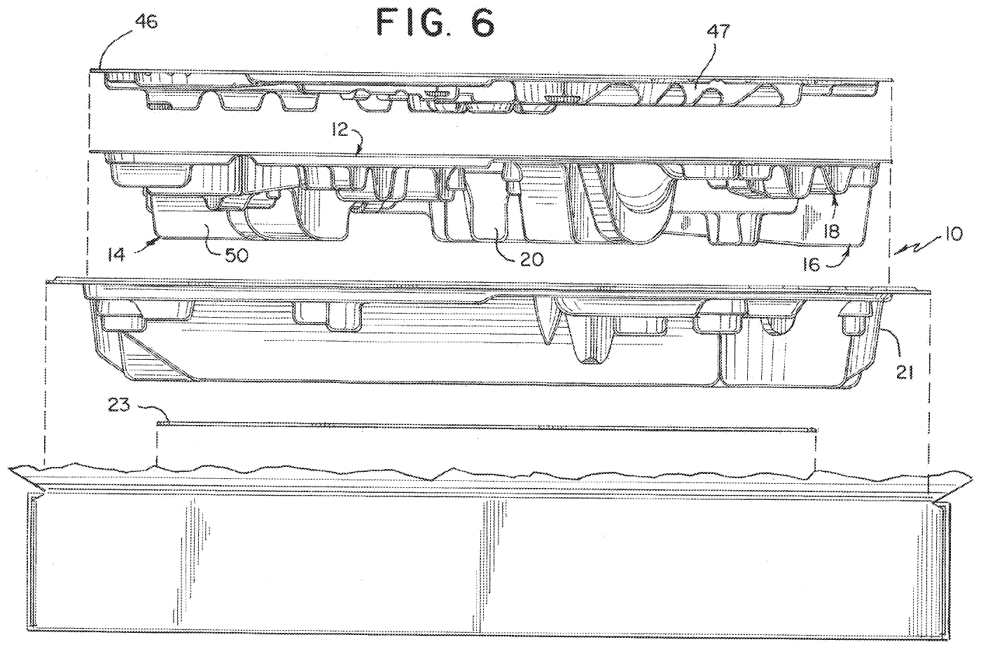

Referring now to FIG. 4, a top view of the retainer cover lid 47 is shown in box 11. Box 11 has two notches 13 to allow the user to place her/his fingers in the box and to remove the retainer cover lid 47, an inner tray 14 and an outer tray 21 from box 11 as shown in FIG. 5. FIG. 6 shows an exploded view of the contents of box 11, namely the flexible tray cover 46, a retainer cover lid 47, an inner tray 12, an outer tray 21, an instruction card 23. It should be noted that retainer cover lid 47 is sealingly attached to outer tray 21 as shown in FIGS. 2, 3 and 6 to seal the contents of the tray 12 in the assembly of the retainer cover lid 47, the inner tray 12 and the outer tray 21. An instruction card 23 is disposed below outer tray 21 inside box 11.

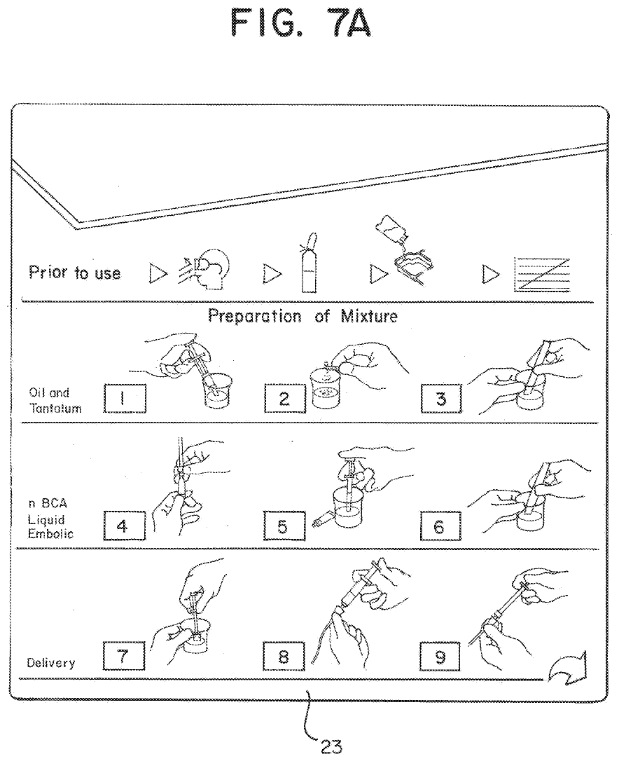

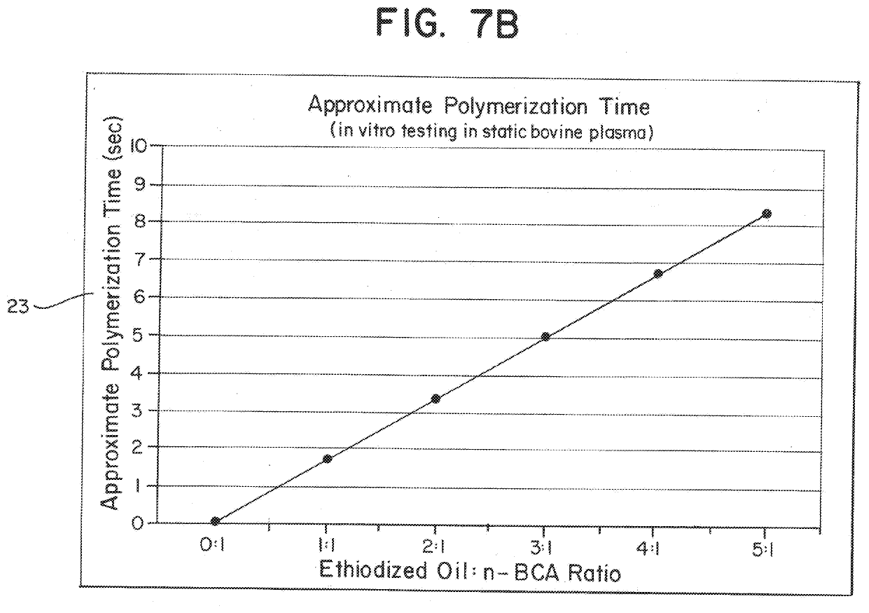

Tray 12 has four sections 14, 16, 18, 19 and a center recess 20 configured to receive a mixing container 22. Mixing container 22 is disposed in the first 14 of the at least three sections of the tray 16, 18, 19. A sealed vial 24 contains an ethiodized oil and is disposed in the first section 14 of tray 12. The ethiodized oil can be, for example, an iodinated poppy seed oil. At least one syringe 26 is disposed in the first section 14 of tray 12. A first needle 28 is disposed in the first section 14 of tray 12. Mixing container 22 is disposed in the first section 14 of tray 12. Instruction card 23 has illustrations and step instructions that match the Instructions For Use (“IFU”) to provide the user with a guide for how to use set 10 on a first side of the instruction card 23 as shown in FIG. 7A. A second side of the instruction card 23 is shown in FIG. 7B which shows the approximate polymerization time in seconds depending upon the ratio of ethiodized oil to n-BCA.

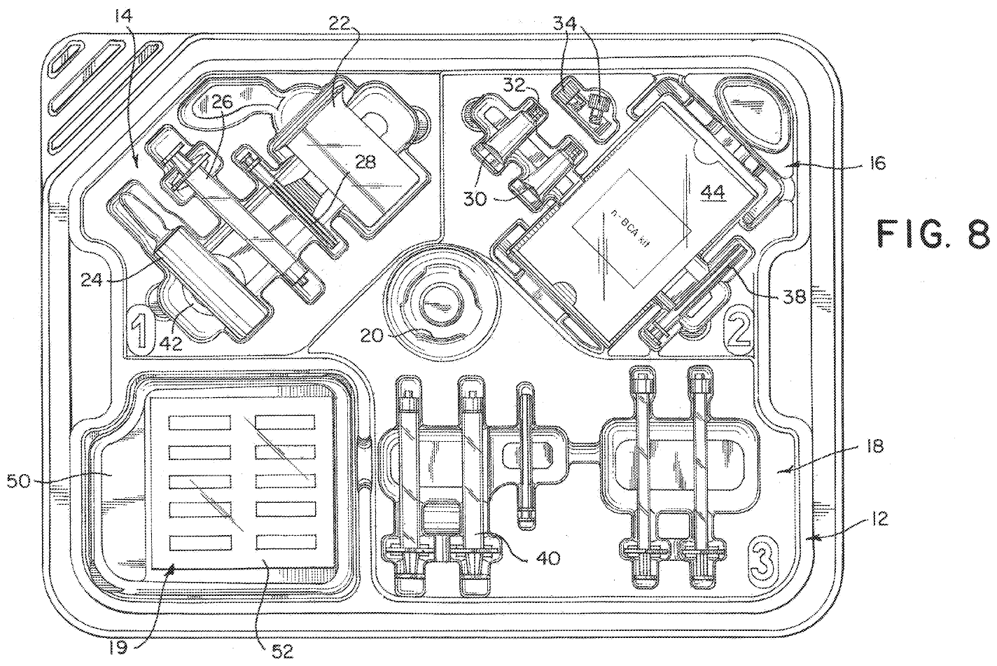

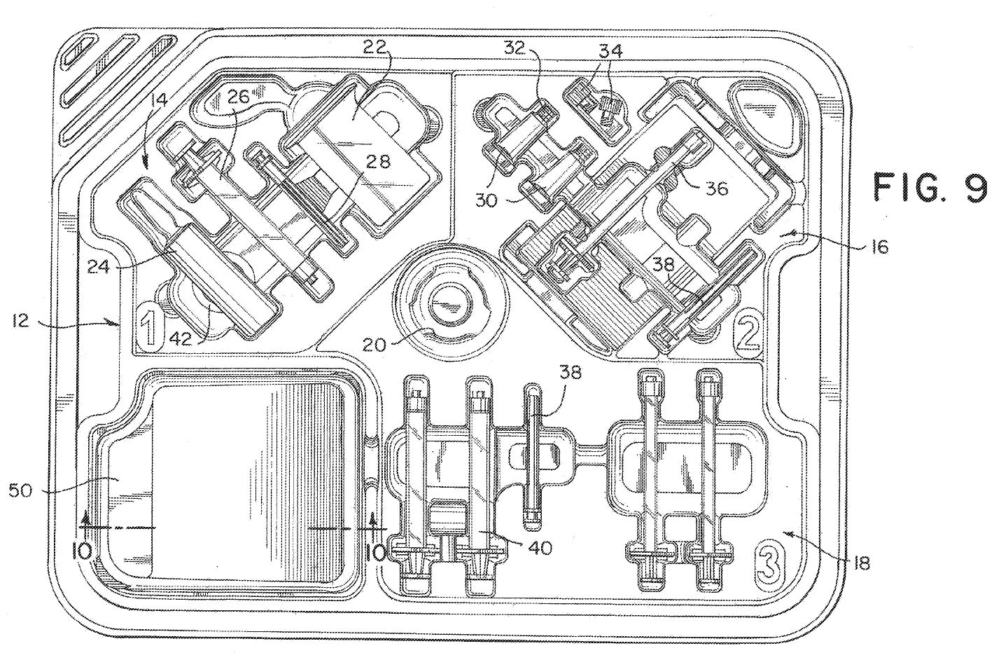

Referring now to FIGS. 8-11, a sealed tube 30 containing a predetermined amount of n-Butyl Cyanoacrylate (“n-BCA”) is disposed in the second section 16 of tray 12. As shown in FIG. 8, two sealed tubes 30 each containing a predetermined amount of n-BCA are disposed in the second section 16 of tray 12. Each sealed tube 30 has an external thread 32 and a sealed cap adjacent the external thread 32. A self-piercing luer cap 34 is disposed in the second section 16 of tray 12. As shown in FIG. 8, two self-piercing luer caps 34 are disposed in the second section 16 of tray 12. Caps 34 each have an internal thread that mates with the external thread 32s of the sealed tubes 30 of n-BCA. A luer lock syringe 36 is disposed in the second section 16 of tray 12 as shown in FIG. 9 below a sealed package 44 that has a container 45 of tantalum (“Ta”) powder disposed inside the sealed package 44.

A second needle 38 and a syringe 40 are disposed in the third 18 section of tray 12. A first cavity 42 in the first section 14 of tray 12 is configured to receive vial 24 of ethiodized oil in an upright position. Vial 24 is stored just above first cavity 42 in tray 12 as shown in FIG. 9. A plurality of syringe labels 52 are disposed in the fourth section 19 of tray 12. The syringe labels 52 can be used to label each syringe each time one is being used.

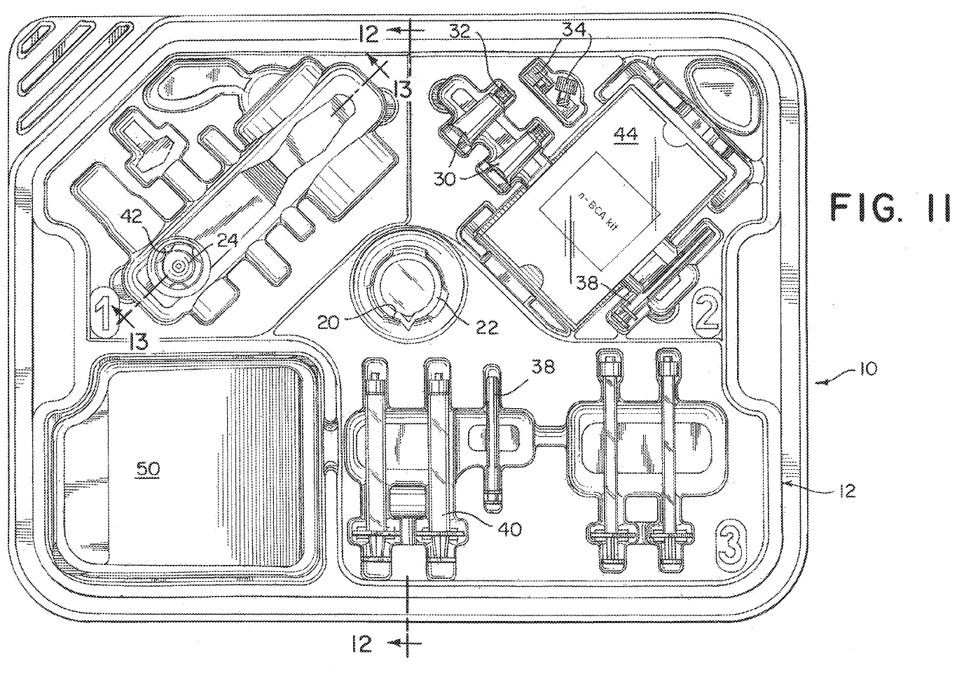

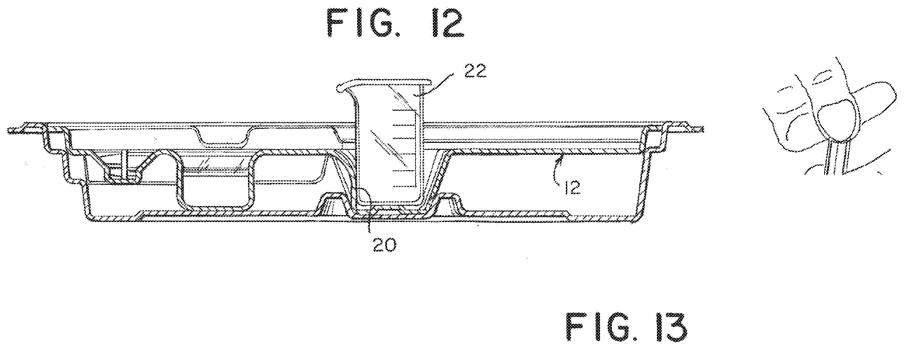

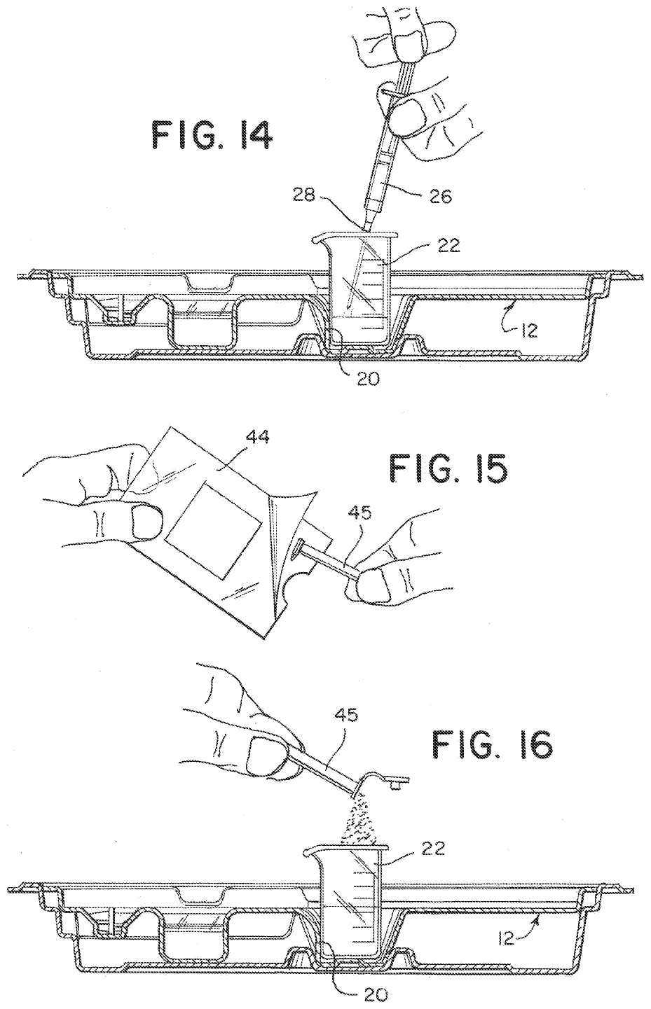

Referring now to FIGS. 3, 5 and 12-20, a method of using the contents of set 10 to prepare a mixture of a predetermined amount of n-BCA and a predetermined amount of an ethiodized oil for treating the vasculature to reduce or block blood flow will be described. The method includes the step of obtaining a set 10 as described above. The user will first open the set by opening box 11 and then removing cover 46 from outer tray 21 as shown in FIGS. 2 and 3. The user can then remove the assembly of retainer cover lid 47, inner tray 12 and outer tray 21 and place in the sterile field. Retainer cover lid 47 can be lifted and removed from the top of inner tray 12 to expose the contents of the inner tray 12. The user can then take vial 24 of ethiodized oil and wipe it clean with, for example, an alcohol swab. The user can then open vial 24 of ethiodized oil using a sterile gauze to snap off the neck of vial 24. Referring now to FIG. 13, vial 24 can be placed upright in receptacle 42 in tray 12. A predetermined amount of the ethiodized oil is withdrawn from vial 24 using first needle 28 and syringe 26 as shown in FIG. 13. Mixing container 22 is placed upright into receptacle 20 in tray 12 as shown in FIG. 12. The withdrawn ethiodized oil is deposited into mixing container 22 as shown in FIG. 14.

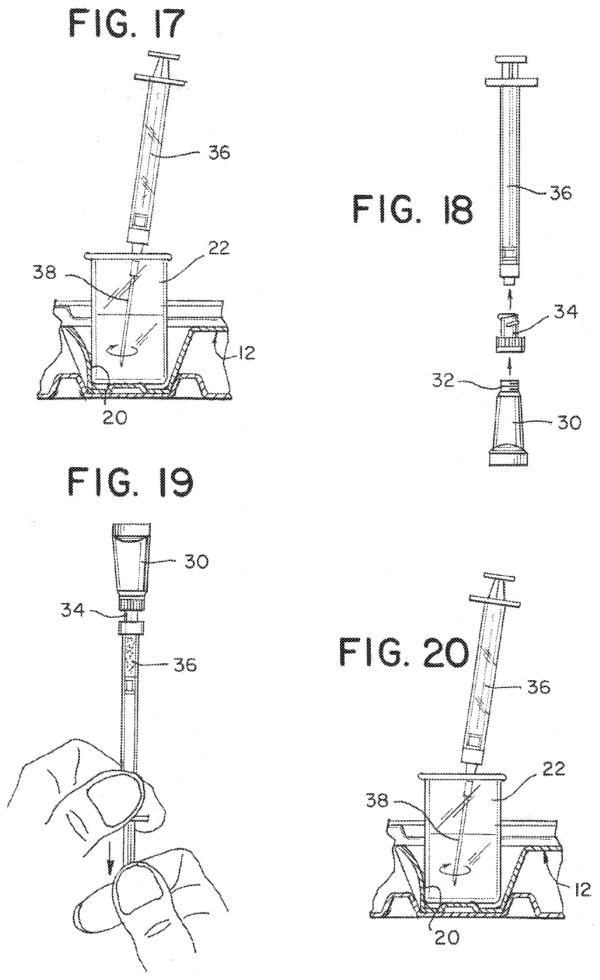

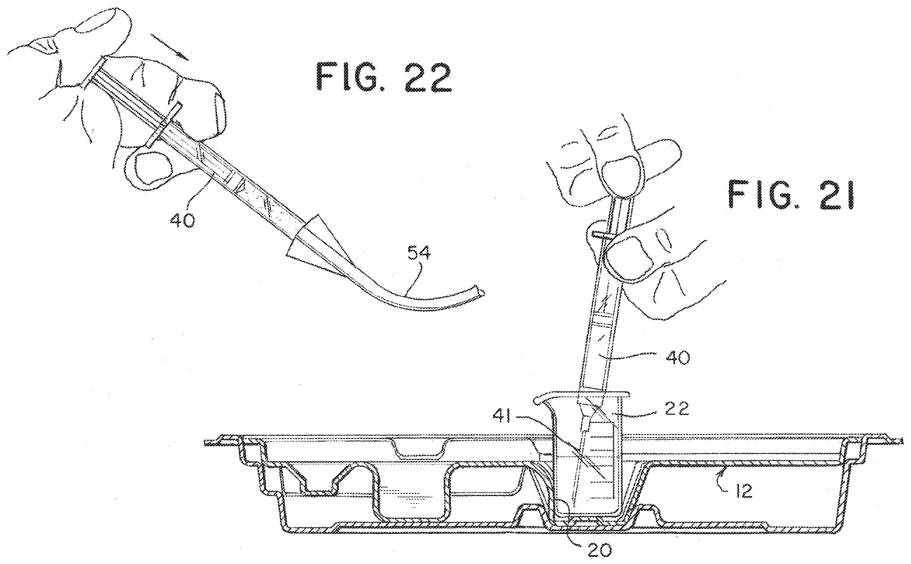

Referring now to FIGS. 18-20, a first end of the self-piercing cap 34 is attached to a syringe 36. A second end of the self-piercing cap 34 is attached to the n-BCA tube 30 with the tube below the self-piercing cap 34 and syringe 36 as shown in FIG. 18. The self-piercing cap 34 is screwed onto tube 30 until resistance is met indicating that the seal on tube 30 has been penetrated. The user will continue screwing cap 34 onto tube 30 until the resistance eases and the seal of the tube 30 is punctured by cap 34, The user will continue screwing cap 34 onto tube 30 until the resistance builds again, indicating a proper seal between the syringe 36, the cap 34 and the tube 30. The syringe 36, cap 34 and tube 30 assembly are turned over so that the tube 30 is above the cap 34 and syringe 36 as shown in FIG. 19. A predetermined amount of the n-BCA is removed from tube 30 and is deposited into syringe 36. Tube 30 and self-piercing cap 34 are removed from the syringe 36. A needle 38 is attached to syringe 36. A predetermined amount of the n-BCA from syringe 36 is added or deposited into the mixing container 22 as shown in FIG. 20. The n-BCA and oil are mixed in the mixing container 22 until the mixture is homogenous. A predetermined amount of the mixture is withdrawn into an injection needle 41 and syringe 40. The user shall then verify that the mixture is well suspended and free of air bubbles.

In some embodiments, the user may add Ta powder to the ethiodized oil before the n-BCA is placed in the mixing container 22. The user will open the sealed package 44 of Ta powder and add the Ta powder into the mixing container 22 as shown in FIGS. 15 and 16. The user will then mix the Ta powder with the oil in the mixing container 22 until the mixture is homogenous as shown in FIG. 17. As shown in FIG. 17, the user can use syringe 36 and needle 38 to mix the oil and Ta powder if desired. Thereafter, the user can follow the steps above to open the tube 30 of n-BCA and mix with the mixed Ta powder and oil in mixing container 22. Then a predetermined amount of the mixture is withdrawn into an injection needle 41 and syringe 40. The user shall then verify that the mixture is well suspended and free of air bubbles.

As shown in FIG. 10, a 5% dextrose solution in water (“D5W”) can be placed in cavity 50 in tray 12 after syringe labels 52 are removed from cavity 19. The user can then prepare a microcatheter 54 by rinsing the outside of a catheter hub and flushing the catheter with the solution of D5W. The homogenous mixture of ethiodized oil, n-BCA and Ta powder, if desired by the user, is then injected through microcatheter 54 to the desired site within the vasculature as shown in FIG. 22. Thereafter, the microcatheter is withdrawn from the vasculature. If desired, the user can aspirate the catheter with the injection syringe before withdrawing the microcatheter from the vasculature step.

After injection is complete, the catheter is immediately withdrawn to prevent adherence of the catheter tip and to ensure that no unpolymerized mixture will leak during catheter withdrawal. The microcatheter and syringe can then be disposed of properly.

By “comprising” or “containing” or “including” is meant that at least the named compound, element, particle, or method or use step is present in the composition or article or method or use, but does not exclude the presence of other compounds, materials, particles, method or use steps, even if the other such compounds, material, particles, method or use steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method or use does not preclude the presence of additional method or use steps or intervening method or use steps between those steps expressly identified. Steps of a method or use may be performed in a different order than those described herein without departing from the scope of the present disclosure. Similarly, it is also to be understood that the mention of one or more components in a device or system does not preclude the presence of additional components or intervening components between those components expressly identified.

The descriptions contained herein are examples illustrating the solution and are not intended to limit the scope. As described herein, the solution contemplates many variations and modifications of a system, device, and/or method or use that can be used to analyze one or more clots and individualize treatment based on the analysis. Variations can include but are not limited to alternative geometries of elements and components described herein, utilizing any of numerous materials for each component or element (e.g. radiopaque materials, memory shape metals, etc.), utilizing additional components, utilizing additional components to perform functions described herein, or utilizing additional components to perform functions not described herein, for example. These modifications would be apparent to those having ordinary skill in the art to which this invention relates and are intended to be within the scope of the claims which follow.

The specific configurations, choice of materials and the size and shape of various elements can be varied according to particular design specifications or constraints requiring a system or method or use constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
1. A set for treating the vasculature to reduce or block blood flow, the set comprising:
   a tray having at least three sections and a center recess configured to receive a mixing container;
   a sealed vial containing an ethiodized oil disposed in a first of the three sections of the tray;
   at least one syringe disposed in one of the at least three sections the tray;
   a first needle disposed in one of the at least three sections the tray;
   a mixing container disposed in a first of the at least three sections of the tray;
   a sealed tube containing a predetermined amount of n-Butyl Cyanoacrylate (“n-BCA”), the sealed tube being disposed in a second of the at least three sections of the tray, the sealed tube having an external thread;
   a self-piercing luer cap disposed in one of the at least three sections of the tray; the cap having an internal thread that mates with the external thread of the sealed tube of n-BCA; and
   a luer lock syringe disposed in one of the at least three sections of the tray.
2. The set according to clause 1, further comprising:
   at least a second needle and syringe disposed in the third of the three sections of the tray.
3. The set according to any of clauses 1-2, further comprising:
   a first cavity in the tray configured to receive the vial of ethiodized oil in an upright position.
4. The set according to any of clauses 1-3, wherein the first cavity in the tray is in the first of the three sections of the tray.
5. The set according to any of clauses 1-4, further comprising:
   a sealed package having a container of tantalum (“Ta”) powder disposed in a second of the three sections of the tray.
6. The set according to any of clauses 1-5, wherein the self-piercing luer cap is disposed in a second one of the at least three sections of the tray.
7. The set according to any of clauses 1-6, further comprising:
   a cover being removably connected to the tray, when the cover is connected to the tray the tray is sealed and the tray contents are configured to be sterilized.
8. The set according to clause 7, wherein the cover is made from a flexible material.
9. The set according to any of clauses 1-8, wherein the ethiodized oil is an iodinated poppy seed oil.
10. The set according to any of clauses 1-9, wherein the sealed vial is an ampule that is made of glass.
11. The set according to any of clauses 1-10, further comprising:
   a second cavity in the tray configured to receive the mixing container in an upright position.
12. The set according to clause 10, wherein the second cavity in the tray is disposed in an approximate center of the tray.
13. The set according to any of clauses 1-12, wherein the mixing container is a glass beaker.
14. The set according to any of clauses 1-13, further comprising:
   a third cavity disposed in the tray, the third cavity is configured to receive a solution of 5% dextrose (“D5W”).
15. The set according to any of clauses 1-14, further comprising:
   a plurality of syringe labels disposed in one of the at least three sections of the tray.
16. The set according to any of clauses 1-15, wherein the needle is an 18G blunt fill needle.
17. The set according to any of clauses 1-16, wherein the needle is a 21G hypodermic needle.
18. A method of preparing a mixture of a predetermined amount of n-Butyl Cyanoacrylate (“n-BCA”) and a predetermined amount of an ethiodized oil for treating treating the vasculature to reduce or block blood flow; the method comprising the steps of:
   obtaining a set, the set comprising:
      a tray having at least three sections and a center recess configured to receive a mixing container;
      a sealed vial containing an ethiodized oil disposed in a first of the three sections of the tray;
      at least one syringe disposed in one of the at least three sections of the tray;
      a first needle disposed in one of the at least three sections of the tray;
      a mixing container disposed in a first the at least three sections of the tray;
      a sealed tube containing a predetermined amount of n-Butyl Cyanoacrylate (“n-BCA”), the sealed tube being disposed in a second of the at least three sections of the tray, the sealed tube having an external thread;
      a self-piercing luer cap disposed in one of the at least three sections of the tray; the cap having an internal thread that mates with the external thread of the sealed tube of n-BCA; and
      a luer lock syringe disposed in one of the at least three sections of the tray;
   opening the vial of ethiodized oil;
   withdrawing a predetermined amount of the ethiodized oil from the vial using the first needle and syringe from the tray;
   placing the mixing container upright into a receptacle in the tray;
   deposit the withdrawn oil into the mixing container;
   attaching a first end of the self-piercing cap to a syringe;
   attaching a second end of the self-piercing cap to the n-BCA tube with the tube below the self-piercing cap and syringe;
   screwing the self-piercing cap onto the tube until a resistance is met, continue screwing the cap onto the tube until the resistance eases and the seal of the tube is punctured by the cap, continue screwing the cap onto the tube until resistance builds again, indicating a proper seal between the syringe, the cap and the tube;
   turn the syringe, cap and tube assembly over so that the tube is above the cap and syringe;
   removing a predetermined amount of the n-BCA from the tube into the syringe;
   removing the tube and self-piercing cap from the syringe;
   attaching a needle to the syringe;
   adding the predetermined amount of the n-BCA from the syringe into the mixing container;
   mixing the n-BCA and oil in the mixing container until the mixture is homogenous;
   withdrawing a predetermined amount of the mixture into an injection needle and syringe; and
   verifying that the mixture is well suspended and free of air bubbles.
19. The method of clause 18, wherein the set further comprises:
   a sealed package having a container of tantalum (“Ta”) powder disposed in a second of the three sections of the tray.
the method further comprising the steps of:
   opening the sealed package of Ta powder;
   adding the Ta powder into the mixing container before the adding the predetermined amount of the n-BCA from the syringe into the mixing container step; and
   mixing the Ta powder with the oil in the mixing container until the mixture is homogenous.
20. The method of any of clauses 18-19, wherein the mixing the Ta powder step includes using the syringe and needle to mix the Ta powder with the n-BCA and oil in the mixing container.
21. The method of any of clauses 18-20, wherein the mixing the n-BCA and oil step included using a needle and syringe from the tray.
22. The method of any of clauses 18-21, wherein the set further comprises:
   a cover being removably connected to the tray, when the cover is connected to the tray the tray is sealed and the tray contents are configured to be sterilized;
the method further comprising the step of:
   before the opening the vial of ethiodized oil step, removing the cover from the tray to expose the contents of the tray to a user.
23. The method of any of clauses 18-22, further comprising the step of:
   cleaning the vial of ethiodized oil with an alcohol wipe before the opening the vial of ethiodized oil step.
24 The method of any of clauses 18-23, further comprising the steps of:
   preparing a microcatheter by rinsing the outside of a catheter hub and flushing the catheter with a solution of D5W;
   injecting the mixture through the microcatheter to the desired site within the vasculature; and
   withdrawing the microcatheter from the vasculature.
25. The method of any of clauses 18-24, further comprising the steps of:
   aspirating with the injection syringe before withdrawing the microcatheter from the vasculature step.

What is claimed is:

1. A set for treating the vasculature to reduce or block blood flow, the set comprising:
a tray having at least three sections and a center recess configured to receive a mixing container;
a sealed vial containing an ethiodized oil disposed in a first of the three sections of the tray;
at least one syringe disposed in one of the at least three sections the tray;
a first needle disposed in one of the at least three sections the tray;
a mixing container disposed in a first of the at least three sections of the tray;
a sealed tube containing a predetermined amount of n-Butyl Cyanoacrylate (“n-BCA”), the sealed tube being disposed in a second of the at least three sections of the tray, the sealed tube having an external thread;
a self-piercing luer cap disposed in one of the at least three sections of the tray; the cap having an internal thread that mates with the external thread of the sealed tube of n-BCA; and
a luer lock syringe disposed in one of the at least three sections of the tray.

2. The set according to claim 1, further comprising:
at least a second needle and syringe disposed in the third of the three sections of the tray.

3. The set according to claim 1, further comprising:
a first cavity in the tray configured to receive the vial of ethiodized oil in an upright position.

4. The set according to claim 2, wherein the first cavity in the tray is in the first of the three sections of the tray.

5. The set according to claim 1, further comprising:
a sealed package having a container of tantalum (“Ta”) powder disposed in a second of the three sections of the tray.

6. The set according to claim 1, wherein the self-piercing luer cap is disposed in a second one of the at least three sections of the tray.

7. The set according to claim 1, further comprising:
a cover being removably connected to the tray, when the cover is connected to the tray the tray is sealed and the tray contents are configured to be sterilized.

8. The set according to claim 7, wherein the cover is made from a flexible material.

9. The set according to claim 1, wherein the ethiodized oil is an iodinated poppy seed oil.

10. The set according to claim 1, wherein the sealed vial is a glass ampule.

11. The set according to claim 1, further comprising:
a second cavity in the tray configured to receive the mixing container in an upright position.

12. The set according to claim 10, wherein the second cavity in the tray is disposed in an approximate center of the tray.

13. The set according to claim 12, wherein the mixing container is a glass beaker.

14. The set according to claim 1, further comprising:
a third cavity disposed in the tray, the third cavity is configured to receive a solution of 5% dextrose (“D5W”).

15. The set according to claim 1, further comprising:
a plurality of syringe labels disposed in one of the at least three sections of the tray.

16. The set according to claim 1, wherein the needle is an 18G blunt fill needle.

17. The set according to claim 1, wherein the needle is a 21G hypodermic needle.

18. A method of preparing a mixture of a predetermined amount of n-Butyl Cyanoacrylate (“n-BCA”) and a predetermined amount of an ethiodized oil for treating treating the vasculature to reduce or block blood flow; the method comprising the steps of:
obtaining a set, the set comprising:
a tray having at least three sections and a center recess configured to receive a mixing container;
a sealed vial containing an ethiodized oil disposed in a first of the three sections of the tray;
at least one syringe disposed in one of the at least three sections of the tray;
a first needle disposed in one of the at least three sections of the tray;
a mixing container disposed in a first the at least three sections of the tray;
a sealed tube containing a predetermined amount of n-Butyl Cyanoacrylate (“n-BCA”), the sealed tube being disposed in a second of the at least three sections of the tray, the sealed tube having an external thread;
a self-piercing luer cap disposed in one of the at least three sections of the tray;
the cap having an internal thread that mates with the external thread of the sealed tube of n-BCA; and
a luer lock syringe disposed in one of the at least three sections of the tray;
opening the vial of ethiodized oil;
withdrawing a predetermined amount of the ethiodized oil from the vial using the first needle and syringe from the tray;
placing the mixing container upright into a receptacle in the tray;
deposit the withdrawn oil into the mixing container;
attaching a first end of the self-piercing cap to a syringe;
attaching a second end of the self-piercing cap to the n-BCA tube with the tube below the self-piercing cap and syringe;
screwing the self-piercing cap onto the tube until a resistance is met, continue screwing the cap onto the tube until the resistance eases and the seal of the tube is punctured by the cap, continue screwing the cap onto the tube until resistance builds again, indicating a proper seal between the syringe, the cap and the tube;
turn the syringe, cap and tube assembly over so that the tube is above the cap and syringe;
removing a predetermined amount of the n-BCA from the tube into the syringe;
removing the tube and self-piercing cap from the syringe;
attaching a needle to the syringe;
adding the predetermined amount of the n-BCA from the syringe into the mixing container;
mixing the n-BCA and oil in the mixing container until the mixture is homogenous;
withdrawing a predetermined amount of the mixture into an injection needle and syringe; and
verifying that the mixture is well suspended and free of air bubbles.

19. The method of claim 18, wherein the set further comprises:
a sealed package having a container of tantalum (“Ta”) powder disposed in a second of the three sections of the tray. the method further comprising the steps of:
opening the sealed package of Ta powder;
adding the Ta powder into the mixing container before the adding the predetermined amount of the n-BCA from the syringe into the mixing container step; and
mixing the Ta powder with the oil in the mixing container until the mixture is homogenous.

20. The method of claim 18, wherein the set further comprises:
a cover being removably connected to the tray, when the cover is connected to the tray the tray is sealed and the tray contents are configured to be sterilized;
the method further comprising the step of:
before the opening the vial of ethiodized oil step, removing the cover from the tray to expose the contents of the tray to a user.
